(19) United States
(12) Patent Application Publication
(10) Pub. No.: US 2017/0049623 A1
(43) Pub. Date: Feb. 23, 2017

(54) PHOTOCOAGULATION DEVICE AND A METHOD THEREOF

(71) Applicant: FORUS HEALTH PVT. LTD., Bangalore, Karnataka (IN)

(72) Inventors: Santosh Kumar COTHURU, Bangalore (IN); Subhash NARAYANAN, Bangalore (IN); Shyam VASUDEVA RAO, Bangalore (IN)

(73) Assignee: FORUS HEALTH PVT. LTD., Bangalore, Karnataka (IN)

(21) Appl. No.: 14/916,412

(22) PCT Filed: Jan. 29, 2015
(86) PCT No.: PCT/IB2015/050671

§ 371 Date: Mar. 3, 2016

(30) Foreign Application Priority Data
Jan. 31, 2014 (IN) 447/CHE/2014

(51) Int. Cl. A61F 9/008

(52) U.S. Cl. CPC A61F 9/00821; A61F 9/00823; A61F 2009/00863

(57) ABSTRACT

Embodiments of the present disclosure provide a photocoagulation device and a method to produce a light beam of a predefined wavelength using the photocoagulation device. The device comprises at least one light source, non-imaging light collimator (NILC), at least one first and second condenser, a ball lens and at least one galvo-mirror. The light source is one of light emitting diode (LED) and organic LED (OLED), which emits light of a predefined wavelength. The NILC collimates the light emitted by the at least one light source, the at least one first condenser produces a focused light beam using the collimated light. The at least one second condenser produces light spots, with a diameter in terms of microns, using the focused light beam from the at least one first condenser. The ball lens collimates the light spots, which is steered by the galvo-mirror to focus on a target area.

### TECHNICAL FIELD

The present disclosure generally relates to a photocoagulation device, and more particularly the present disclosure relates to photocoagulation device using light emitting diodes (LEDs).

### BACKGROUND

Presently, delivery of intense light at required wavelengths to tightly focused regions of the retina, for the purpose of photocoagulation of retinal blood vessels is one of the core technical problems. The choice of intensities, wavelengths and focus areas are determined by medical research and the problems presented by a particular patient.

In the recent times, photocoagulation uses expensive lasers, and is widely utilized to treat a variety of retinal diseases, such as proliferative diabetic retinopathy (DR), diabetic macular oedema (DMO), retinopathy of prematurity (ROP), retinal vein occlusions, and retinal tears. The photocoagulation is the prescribed as the first-choice intervention for DR. Laser-based photo-coagulators are used for treatment of DR. The photocoagulation use high power lasers to spot weld and seal leakage areas in the retina, remove/eliminate abnormal blood vessels produced by neovascularization, and treat peripheral retina involved in vascular endothelial growth through pan retinal photocoagulation.

The currently available photocoagulators adapt to slit lamps and head mounted delivery systems. These photocoagulators provide tightly focused (down to 50 microns) multiple spots to the treatment site/lesion for precise targeting of the affected area. This is often performed in programmed scan patterns to reduce treatment time and improve accuracies. Also, the photo-coagulators provide multi-wavelength options to facilitate treatment of various retinal diseases. The lasers used for photocoagulation have continuous wave (CW) output power in the range of 0-2000 mW, and can be operated also in the pulsed mode with pulse widths in the range of 10-3000 ms. The lasers may be focused to spot sizes of 50-500 μm for efficient photocoagulation. However, these photo-coagulators are very costly and bulky for transport to remote areas.

Known in the art are new developments in photocoagulation systems, which are propelled by the desire for better visual outcomes with reduced side effects and treatment costs. The objective is to produce high precision burns with minimal damage to surrounding tissues. All existing devices for photocoagulation use lasers, as these emit high power monochromatic light that can be focused to tight spot sizes of 50-500 microns. Furthermore, they are available at wavelengths matching the absorption of major retinal tissue absorbers. Nonetheless, the existing systems used against retinal diseases are very costly as they use lasers such as ‘second harmonic of Q-switched Nd: YAG’ at 532 nm, ‘argon laser’ at 488 nm, ‘krypton laser’ at 647 nm and the recent ‘diode pumped solid state (DPSS) laser’ at 577 nm, matching with the absorption of oxygenated hemoglobin. Furthermore, most of the advanced photo-coagulators available in the market integrate multiple lasers to adapt a single system for treatment of various retinal diseases, thereby raising the total cost further.

Accordingly, a need exists for a device and a method to generate a light beam of predefined wavelength by reducing device cost and size, thus, making the device less complex, portable and affordable.

### SUMMARY

One or more shortcomings of the prior art are overcome and additional advantages are provided through the present disclosure. Additional features and advantages are realized through the techniques of the present disclosure. Other embodiments and aspects of the disclosure are described in detail herein and are considered a part of the claimed disclosure.

The present disclosure provides a photocoagulation device comprising at least one light source, non-imaging light collimator (NILC), at least one first and second condenser, a ball lens and at least one galvo-mirror. The light source is one of light emitting diode (LED) and organic LED (OLED), which emits light of a predefined wavelength. The NILC collimates the light emitted by the at least one light source, the at least one first condenser produces a focused light beam using the collimated light. The at least one second condenser produces light spots, with a diameter in terms of microns, using the focused light beam from the at least one first condenser. The ball lens collimates the light spots, which is steered by the galvo-mirror to focus on a target area.

Further, the present disclosure provides a method to produce a light beam of a predefined wavelength for photocoagulation. The method comprises generating light emission from the light source, of a predefined wavelength by controlling temperature across at least one light emitting diode (LED) of the light source. Also the method comprises, collimating light emitted from the at least one LED by at least one non-imaging light collimator (NILC). Further, the method comprises converging of the collimated light by at least one condenser to form a focused light beam. Furthermore, the method comprises producing light spots of a few microns in diameter, by at least a second condenser from the focused light beam. Thereafter, the method comprises steering the light spots by at least one galvo-mirror, for focusing on a target area. The light spots produced by the at least one second condenser are collimated using a ball lens, which are steered by the galvo-mirror. The light spot size is varied by a beam modification block (BMB), which is coupled to the at least one galvo-mirror.

The foregoing summary is illustrative only and is not intended to be in any way limiting. In addition to the illustrative aspects and features described above, further aspects, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1A illustrates an exemplary block diagram of a photocoagulation device, in accordance with an embodiment of the present disclosure;

FIG. 1B illustrates a photocoagulation device in accordance with some embodiments of the present disclosure;

FIG. 2 illustrates an LED wavelength steering embodiment in accordance with an embodiment of the present disclosure;

FIG. 3A illustrates a light source of a photocoagulation device comprising plurality of LEDs arranged radially around a rotating mirror in accordance with an alternative embodiment of the present disclosure;

FIG. 3B shows a graph illustrating a sequence of turning on each individual LED's of the photocoagulation device of FIG. 3A, in accordance with an embodiment of the present disclosure;

FIG. 4 illustrates a photocoagulation device in accordance with an alternative embodiment of the present disclosure; and

FIG. 5 illustrates a plot showing absorption spectra of ocular pigments and a Light Emitting Diode (LED) of the photocoagulation device in accordance with some embodiments of the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific aspect disclosed may be readily utilized as a basis for modifying or designing other structures for carrying out the same purposes of the present disclosure.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or apparatus.

Embodiments of the present disclosure relate to a photocoagulation device for generating a focused light beam of predefined wavelength. The device comprises at least one light source, non-imaging light collimator (NILC), at least one first and second condenser, a ball lens and at least one galvo-mirror. The light source is one of light emitting diode (LED) and organic LED (OLED), which emits light of a predefined wavelength. The NILC collimates the light emitted by the at least one light source, the at least one first condenser produces a focused light beam using the collimated light. The at least one second condenser produces light spots, with a diameter in terms of microns, using the focused light beam from the at least one first condenser. The ball lens collimates the light spots, which is steered by the galvo-mirror to focus on a target area.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

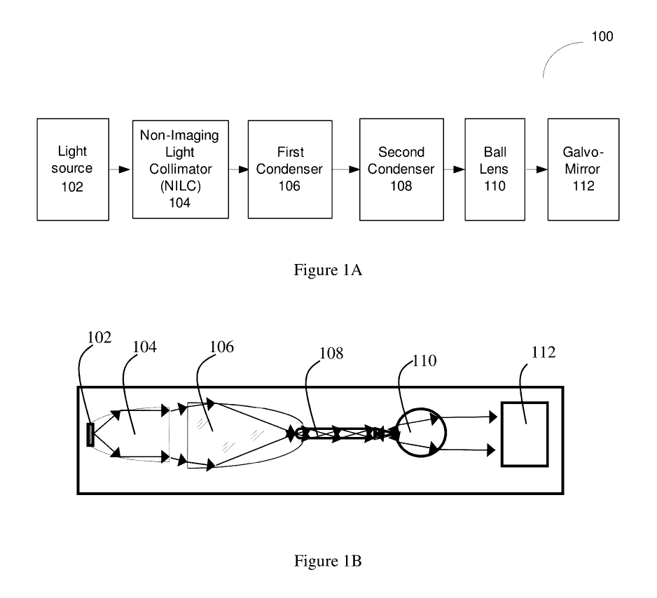

FIG. 1A illustrates an exemplary block diagram of a photocoagulation device, in accordance with an embodiment of the present disclosure.

As shown in FIG. 1A, the photocoagulation device 100 includes at least one light source 102, non-imaging light collimator (NILC) 104, at least one first condenser 106, at least one second condenser 108, a ball lens 110 and at least one galvo-mirror 112. In one embodiment, as shown in FIG. 1A, the at least one light source 102 is a single light source. The light source is one of light emitting diode (LED) and organic LED (OLED). The output light produced from the LED light source or referred as LED source 102 and its associated wavelength is varied/adjusted by varying junction temperature of the LED source. In one embodiment, the LED source 102 is used for treatment of diabetic retinopathy (DR) for which the wavelength of the light emitted is varied. Also, the light emitted by the LED source is tuned for focusing at around 577 nm wavelength, in accordance with an embodiment of the present disclosure.

The NILC 104 is configured to receive the light emitted by the LED light source 102 and collimate the light. In one embodiment, the NILC 104 collimates the light received from the LED light source 102 based on the principle of total internal reflection. The at least one first condenser 106 receives the collimated light from the NILC and produces a focused light beam. Thereafter, the light beam is refocused to tight spots of 1000 microns by using condenser optics into a second condenser or a tapered fiber. In one embodiment, the condenser 106 is non-imaging total internal reflection (TIR) condenser optics. The tapered fiber is coupled to the at least one first condenser 106 using an optical cement to improve collection efficiency of the light beam.

The at least one second condenser 108 receives the focused light beam from the at least one first condenser, to produce light spots with a diameter in terms of microns. In one embodiment, the second condenser is a tapered fiber. The tapered fiber has a hemispherical dome at the input end is immersed in a high refractive condenser using optical cement. This helps in collecting light from wide incidence angles. The other end of the tapered fiber is lensed/tapered to produce 50 micron spot sizes with high optical efficiency. In one embodiment, using optical techniques the photocoagulation device may be operated in continuous mode or pulse mode to produce various spot sizes, and spot pattern scans with the help of a galvo-mirror.

In one embodiment, the tapered fiber comprises a hemispherical dome at the input end is immersed in a high refractive condenser using optical cement. The dome shape of the tapered fiber facilitates collecting the light from wide incidence angles. The other end of the tapered fiber is lensed/tapered to produce 50 micron spot sizes with high optical efficiency. In one embodiment, the device may be operated in one of continuous mode and pulse mode to produce various spot sizes, and spot pattern scans using galvo-mirrors.

The ball lens 110, may be configurable in the photocoagulation device 100, to collimate the light spots received from the at least one second condenser or the tapered fiber 108. The at least one galvo-mirror 112, is configured to steer the collimated light, received from the ball lens to focus on a target area. The galvo-mirror 112 is coupled to a beam modification block (BMB) (not shown in the figure), which is configured to perform one of vary the light spots propagation direction and intensity. The at least one galvo-mirror is a two axis galvo-mirror. The at least one galvo-mirror 112 produces a predefined scan pattern of light that is transferred to the BMB using at least one scanning lens. In one embodiment, the BMB comprises plurality of optical lenses. The plurality of optical lenses comprises at least one of focusing lens, collimating lens, moving lens and beam expander lens.

FIG. 1B illustrates a photocoagulation device, in accordance with some embodiments of the present disclosure. As shown in FIG. 1B, the device comprises a light source 102, non-imaging light collimator (NILC) 104, first condenser 106, second condenser or tapered fiber 108, a ball lens 110 and a galvo-mirror 112. The light source 102 is one of light emitting diode (LED), organic LED (OLED) and semiconductor light source. In one embodiment, the LED light that is incoherent and divergent in nature is focused to a small spot diameter on a target area. A non-imaging technique is used, so that a yellow light from the LED is focused to one or more spots, each spot is in terms of 50 microns. The focusing is achieved by collimating the light using a non-imaging collimator based on total internal reflection [TIR]. Thereafter, the collimated light is refocused to tight spots of 1000 microns by using another non imaging TIR condenser optics into a tapered fiber.

In one embodiment, the photocoagulation device is used in treatment of retinal diseases. The photocoagulation device provides treatment of diseases such as, but not limited to, diabetic retinopathy (DR) and other retinal diseases, using light emitting diodes (LED). The LEDs may be available at fixed wavelengths and with low cost. In one embodiment, macular xanthophyll has greater absorption of blue light than of any other wavelength. The melanin has excellent absorption at all wavelengths, haemoglobin has good absorption in the visible region and when oxygenated has strong absorption in the yellow, at 577 nm, a wavelength that has been found to be very effective for photocoagulation. In one embodiment, the LEDs have larger line width of approximately 20 nm full width at half maximum (FWHM) and divergence when compared to laser sources and hence difficult to focus to small spot sizes. Hence, the LEDs are used instead of lasers which reduce the total system cost and size, enabling the entire assembly to be bundled into a small form factor enabling portability and affordability.

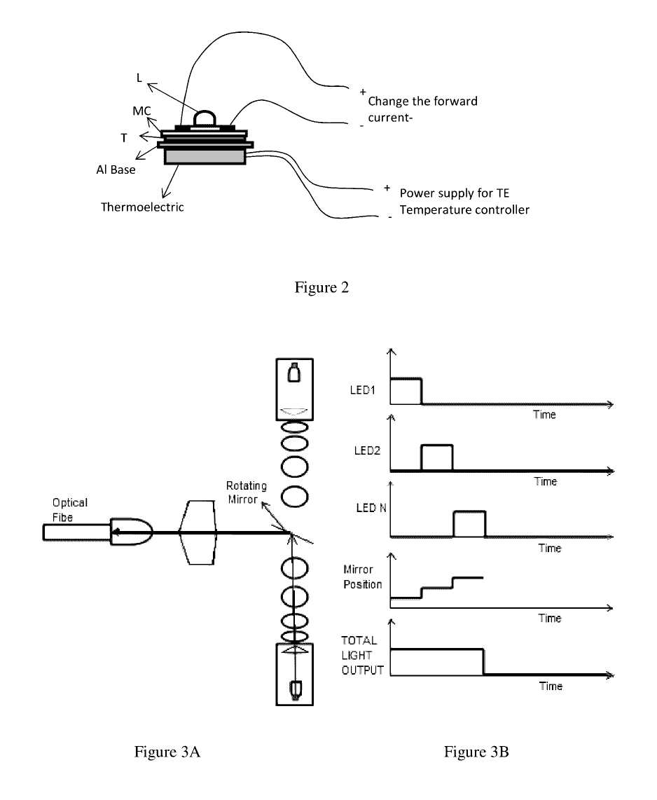

One embodiment of the present disclosure is wavelength tuning of LED. An electro-thermal method is used to tune the output of light emitting diodes (LED). In a monochromatic LED, the dominant wavelength in the LED spectrum increases with temperature. Thus, tuning is of LED to a particular wavelength is possible by adjusting the junction temperature of the LED. The wavelength is adjusted by performing one of controlling the ambient temperature of LED and by varying the forward current through the LED. In an exemplary embodiment, both these ways may be implemented to achieve the targeted shift in wavelength, which is as shown in FIG. 2. The FIG. 2 illustrates an LED with a method for wavelength steering in accordance with an embodiment of the present disclosure.

FIG. 3A illustrates a light source of a photocoagulation device comprising plurality of LEDs arranged radially around a rotating mirror in accordance with an alternative embodiment of the present disclosure. The plurality of LEDs around a rotating mirror rotates circularly to direct light received from each of the plurality of LEDs during ON state to the NILC. Each of the plurality of LEDs is operated at a predefined duty cycle. The rotating mirror collects light beam from an LED at time in a sequential format to provide peak power. Each of the plurality of LED is operated at a duty cycle to provide increased peak power compared to the duty cycle of a conventionally operated LED.

In one embodiment of the present disclosure, as the LEDs are limited by the total power they can emit, an array of LEDs is arranged radially about a central rotating mirror. Each of the LEDs may be pulsed at a high electrical input power for a short duration for switching ON each of the LEDs. The central rotating mirror collects light from each LED during its “ON” period, and each LED may cool during its “OFF” period. By turning only one LED “ON” at a time in a sequential manner as indicated in FIG. 3B, each LED operates at a low duty cycle enabling it to operate at a much increased peak power. FIG. 3B shows a graph illustrating a sequence of turning on each individual LED's of the photocoagulation device of FIG. 3A. By efficiently collecting the light from each LED into a common optical fiber, the total brightness may be increased by many folds, compared to using single LED. This method may be used with any LED, organic LED (OLED) and semiconductor light source, and provides multifold increase in brightness against static single LED packages.

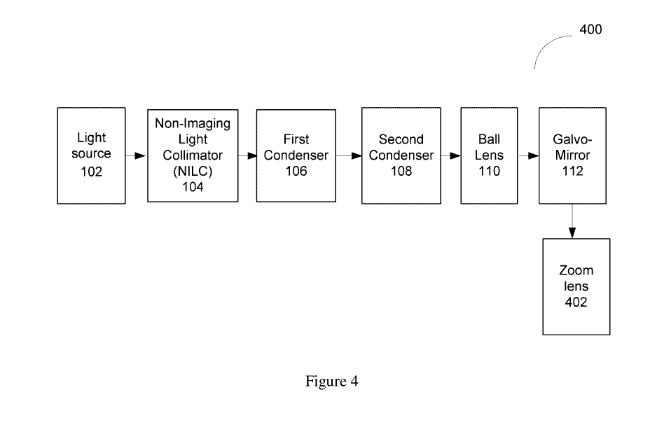

FIG. 4 illustrates a photocoagulation device in accordance with an alternative embodiment of the present disclosure. As shown in FIG. 4, the photocoagulation device 100 includes at least one light source 102, non-imaging light collimator (NILC) 104, at least one first condenser 106, at least one second condenser 108, a ball lens 110, at least one galvo-mirror 112 and zoom lens 402. The at least one light source is one of light emitting diode (LED) and organic LED (OLED). The output light produced from the LED light source or referred as LED source 102 and its associated wavelength is varied/adjusted by varying junction temperature of the LED source. The NILC 104 is configured to receive the light emitted by the LED light source 102 and collimate the light, based on the principle of total internal reflection. The at least one first condenser 106 receives the collimated light from the NILC and produces a focused light beam. Thereafter, the light beam is refocused to tight spots of 1000 microns by using condenser optics into a second condenser or a tapered fiber 108.

In one embodiment, the condenser 106 is non-imaging total internal reflection (TIR) condenser optics. The tapered fiber 108 is coupled to the at least one first condenser 106 using an optical cement to improve collection efficiency of the light beam. In one embodiment, using optical techniques the photocoagulation device may be operated in continuous mode or pulse mode to produce various spot sizes, and spot pattern scans with the help of a galvo-mirror. The ball lens 110, may be configurable in the photocoagulation device 100, to collimate the light spots received from the at least one second condenser or the tapered fiber 108. The at least one galvo-mirror 112, is configured to steer the collimated light, received from the ball lens to focus on a target area.

In one embodiment, an input end of the tapered fiber 108 is immersed in a high refractive index (n) non-imaging condenser using optical cement, which improves the collection efficiency of the light by n². The other end of the tapered fiber is lensed/tapered to produce 50 micron spot sizes and the light is collimated using one of the ball lens or by any other means which is suitable, the collimated light is steered by the 2 axis galvo scanner or mirror to produce desired scan pattern on the retina, the reflected light from the 2 axis galvo mirror is focused by the scanning lens to transfer the scan pattern in to the zoom lens unit or system 402 which is used to adjust the laser spot in a range of 50 microns to 500 microns. The light from the 2 axis scanner is coupled into the zoom system 402, which comprises a focusing lens L1 and a lens L2 (not shown in the figure), is a collimating lens configured to collimate the light and make the light parallel. The zoom lens system 402 also comprises lens L3, L4 and L5 for compromising beam expander that may zoom. In one embodiment, the lens L3, L4 are moving lens, configurable for varying beam expander amplification ratio and change light spot diameter size. Further, the zoom lens system 402 comprises a lens L6, which is a focusing lens for focusing the light from the lens L5.

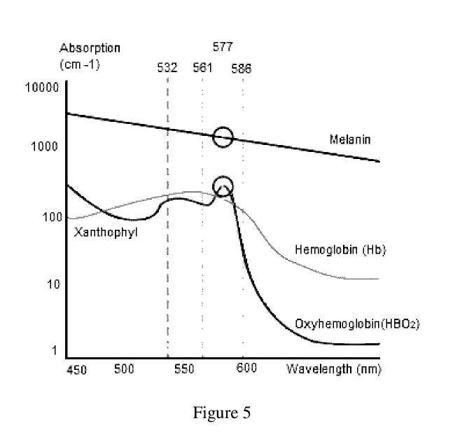

FIG. 5 illustrates a plot showing absorption spectra of ocular pigments and a Light Emitting Diode (LED) of the photocoagulation device in accordance with some embodiments of the present disclosure. Embodiments of the present disclosure provide a solution for photocoagulation treatment of diabetic retinopathy (DR) and other retinal diseases using low-cost light emitting diodes (LED). The LEDs may be available at fixed wavelengths, but these rarely match specific absorption peaks of the various ocular pigments, as shown in FIG. 5. Macular xanthophyll has greater absorption of blue light than of any other wavelength. Melanin has excellent absorption at all wavelengths. Haemoglobin has good absorption in the visible region, and oxygenated has strong absorption in the yellow, at 577 nm, a wavelength that has been found to be very effective for photocoagulation.

In one embodiment, the advantages of photocoagulation device with longer wavelength LEDs is at least one of lesser scattering than shorter wavelength light and providing sharper focusing; more tissue penetration and lesser energy requirement; high oxy-haemoglobin to melanin absorption ratio (effective for vascular structures), and negligible absorption by macular xanthophyll (allowing treatment close to the fovea).

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The terms “a”, “an” and “the” mean “one or more”, unless expressly specified otherwise.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The foregoing description of various embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims hereinafter appended.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

Referral Numerals:

| Reference | |
|---|---|
| Number | Description |
| 100 | photocoagulation device |
| 102 | light source |
| 104 | non-imaging light collimator (NILC) |
| 106 | first condenser |
| 108 | second condenser or tapered fiber |
| 110 | ball lens |
| 112 | galvo-mirror |
| 402 | zoom lens |

1. A photocoagulation device, comprising:
at least one light source to emit light of a predefined wavelength, wherein wavelength of the light beam is adjusted by varying junction temperature of the light source;
at least one non-imaging light collimator (NILC) to collimate the light emitted by the at least one light source;
at least one first condenser to produce a focused light beam using the collimated light received from the NILC;
at least one second condenser to produce light spots, with a diameter in terms of microns, using the focused light beam received from the at least one first condenser;
a ball lens to collimate the light spots received from the at least one second condenser; and
at least one galvo-mirror to steer the collimated light, received from the ball lens to focus on a target area.

2. The device as claimed in claim 1, wherein the at least one light source is one of light emitting diode (LED) and organic LED (OLED).

3. The device as claimed in claim 1, wherein the galvo-mirror is coupled to a beam modification block (BMB), which is configured to perform one of vary the light spots propagation direction and intensity.

4. The device as claimed in claim 3, wherein the BMB comprises plurality of optical lenses.

5. The device as claimed in claim 4, wherein the plurality of optical lenses comprises at least one of focusing lens, collimating lens, moving lens and beam expander lens.

6. The device as claimed in claim 3, wherein the spot size of light beam is varied to generate visible coagulation in at least one region of the target plane.

7. (canceled)

8. The device as claimed in claim 1, wherein the light source comprising plurality of LEDs arranged circumferentially in a ring shape, around a rotating mirror, which rotates circularly to direct light received from each of the plurality of LEDs during ON state to the NILC.

9. The device as claimed in claim 8, wherein each of the plurality of LEDs is operated at a predefined duty cycle.

10. The device as claimed in claim 8, wherein the rotating mirror collects light beam from an LED at a time in a sequential format to provide peak power.

11. The device as claimed in claim 8, wherein each of the plurality of LED is operated at a duty cycle to provide increased peak power compared to the duty cycle of a conventionally operated LED.

12. The device as claimed in claim 1, wherein the at least one NILC collimates light based on the principle of total internal reflection.

13. The device as claimed in claim 1, wherein the at least one second condenser is a tapered fiber.

14. The device as claimed in claim 13 wherein the tapered fiber is coupled to at least one first condenser to improve collection efficiency of the light beam.

15. The device as claimed in claim 1, wherein the at least one galvo-mirror is a two axis galvo-mirror.

16. The device as claimed in claim 1, wherein the at least one galvo-mirror produces a predefined scan pattern of light that is transferred to the BMB using at least one scanning lens.

17.-19. (canceled)

20. The device as claimed in claim 3, wherein the at least one galvo-mirror produces a predefined scan pattern of light that is transferred to the BMB using at least one scanning lens.
